(19) United States
(12) Patent Application Publication
(10) Pub. No.: US 2002/0177616 A1
(43) Pub. Date: Nov. 28, 2002

(54) Use of chromans

(76) Inventors: Thomas Fahrig, Bergisch Gladbach (DE); Irene Gerlach, Koln (DE); Ervin Horvath, Leverkusen (DE); Reinhard Jork, Haan (DE); Frank Mauler, Overath (DE)

Correspondence Address: Jeffrey M. Greenman, Vice President, Patents and Licensing, Bayer Corporation, 400 Morgan Lane, West Haven, CT, 06516, US

(21) Appl. No.: 10/045,884

(22) Filed: Jan. 9, 2002

(30) Foreign Application Priority Data
Jan. 16, 2001 (DE) 10101917.3

(51) Int. Cl. A61K 31/425

(52) U.S. Cl. 514/373

(57) ABSTRACT

The invention relates to the use of 2-[4-({[(2R)-8-isopropoxy-chroman-2-yl]-methyl}amino)butyl]-1,2-benzisothiazol-3(2H)-one 1,1-dioxide, its physiologically acceptable salts, hydrates and/or solvates, in particular its hydrochloride, for the production of a medicament for the prophylaxis and/or treatment of Parkinson's disease.

The invention relates to the use of 2-[4-({[(2R)-8-isopropoxy-chroman-2-yl]methyl}amino)butyl]-1,2-benzisothiazol-3(2H)-one 1,1-dioxide, its physiologically acceptable salts, hydrates and/or solvates, in particular its hydrochloride, for the production of a medicament for the prophylaxis and/or treatment of Parkinson's disease.

Parkinson's disease is a chronic, progressive condition of the central nervous system. It is caused by the degeneration of dopaminergic neurons in the substantia nigra, which produce and release the neurotransmitter dopamine. The decrease in the dopaminergic neurotransmission resulting therefrom leads to massive dysfunctions of the extrapyramidal system of motor control. These disorders relate not only to the basal ganglia but also to other closely linked areas of the brain.

The aetiology of idiopathic Parkinsonism is still largely unknown. Increasing evidence points to the fact that cell death of dopaminergic neurons of the substantia nigra comes about due to apoptosis as a result of mitochondrial dysfunctions. In addition to possible genetic disorders, raised glutamate levels and/or a deficient supply of neurotrophic factors are also discussed as a cause of the mitochondrial dysfunctions.

Starting from this, further progressive neuronal cell death is supposed to be prevented by neuroprotective pharmalogical influencing of the neurodegenerative processes, by which the progression of the condition could be stopped without necessarily interacting with the causal pathophysiological mechanisms.

It has been shown that stimulation of neuronal 5-HT_{1A} receptors in various in vitro and in vivo systems has both neuroprotective, and anti-apoptotic and neurotrophic effects. Stimulation of 5-HT_{1A} receptors could accordingly also prevent the further degeneration of dopaminergic neurons in Parkinson's disease and in the course of this ultimately delay the progression of the condition.

The therapeutics currently used clinically for Parkinson's disease in the majority follow a purely symptomatic approach. The aim of these therapies is either direct substitution of the lacking dopamine by a dopamine precursor molecular (L-DOPA), which is metabolized in the body to dopamine, or else stimulation of deficient dopaminergic neurotransmission processes by means of agonists on dopamine receptors or by decreasing the breakdown of dopamine (MAO inhibitors, COMT inhibitors). All current therapies, however, are characterized by severe side effects (e.g. dyskinesia, psychoses, sleep disturbances) or long-term loss of action.

Chroman derivatives and especially 2-[4-({[(2R)-8-isopropoxy-chroman-2-yl]methyl}amino)butyl]-1,2-benzisothiazol-3(2H)-one 1,1-dioxide hydrochloride and its agonistic action on the 5-HT_{1A} receptor have been disclosed as a means for the treatment of conditions of the central nervous system in EP-A-0 352 613 and EP-A-0 749 970.

WO 99/26621 describes chroman derivatives, in particular 2-[4-({[(2R)-chroman-2-yl]methyl}amino)butyl]-1,2-benzisothiazol-3(2H)-one 1,1-dioxide hydrochloride (generic name: repinotan hydrochloride), as a means for promoting neuroregeneration in neurological conditions such as, for example, Parkinson's disease.

Surprisingly, with 2-[4-({[(2R)-8-isopropoxy-chroman-2-yl]methyl}amino)butyl]-1,2-benzisothiazol-3(2H)-one 1,1-dioxide a 5-HT_{1A} receptor agonist has now been found which not only has a neuroprotective action, but additionally also has a symptomatic activity and thus positively influences the course of Parkinson's disease in a dual manner.

The invention therefore relates to the use of 2-[4-({[(2R)-8-isopropoxy-chroman-2-yl]methyl}amino)butyl]-1,2-benzisothiazol-3(2H)-one 1,1-dioxide, its physiologically acceptable salts, hydrates and/or solvates, in particular 2-[4-({[(2R)-8-isopropoxy-chroman-2-yl]methyl}amino)butyl]-1,2-benzisothiazol-3(2H)-one 1,1-dioxide hydrochloride, for the production of a medicament for the prophylaxis and/or control of Parkinson's disease.

2-[4-({[(2R)-8-Isopropoxy-chroman-2-yl]methyl}amino)butyl]-1,2-benzisothiazol-3(2H)-one 1,1-dioxide has the following structure:

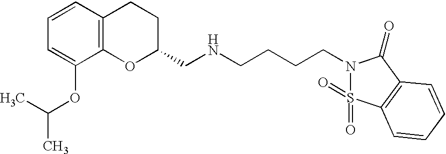

Physiologically acceptable salts of the compounds used according to the invention can be salts of the compounds with mineral acids, carboxylic acids or sulphonic acids. Particularly preferred salts are those, for example, with hydrochloric acid, hydrobromic acid, sulphuric acid, phosphoric acid, methanesulphonic acid, ethanesulphonic acid, toluenesulphonic acid, benzenesulphonic acid, naphthalenedisulphonic acid, acetic acid, propionic acid, lactic acid, tartaric acid, oxalic acid, citric acid, fumaric acid, maleic acid or benzoic acid.

Hydrates within the meaning of the invention are stoichiometric compositions of 2-[4-({[(2R)-8-isopropoxy-chroman-2-yl]methyl }amino)butyl]-1,2-benzisothiazol-3(2H)-one 1,1-dioxide or its salts with water.

Solvates within the meaning of the invention are stoichiometric compositions of 2-[4-({[(2R)-8-isopropoxy-chroman-2-yl]methyl}amino)butyl]-1,2-benzisothiazol-3(2H)-one 1,1-dioxide or its salts with solvents.

The compounds used according to the invention can be prepared by the processes specified in EP-A-0 749 970. For example: 2-[4-({[(2R)-8-isopropoxy-chroman-2-yl]methyl}amino)butyl]-1,2-benzisothiazol-3(2H)-one 1,1-dioxide hydrochloride (called Example 11 below) corresponds to Example 7 in EP-A-0 749 970.

The salts of 2-[4-({[(2R)-8-isopropoxy-chroman-2-yl]methyl}amino)butyl]-1,2-benz-isothiazol-3(2H)-one 1,1-dioxide can be obtained by reacting the free base in a suitable solvent with stoichiometric or superstoichiometric amounts of the acid on which the salt is based in a temperature range from 0° C. up to the boiling point of the solvent. Suitable solvents are, for example, water, aliphatic alcohols such as methanol, ethanol or 2-propanol, aliphatic open-chain or cyclic ethers such as diethyl ether, tert-butyl methyl ether, dioxane, tetrahydrofuran or aliphatic ketones such as 2-propanone, 2-butanone, and their mixtures. The salts are obtained directly from this mixture, if appropriate after partially or completely distilling off the solvent, as a solid; they can be purified by recrystallization or reprecipitation in, for example, the abovementioned solvents or their mixtures.

The active compound can act systemically and/or locally. For this purpose, it can be administered in a suitable manner, such as, for example, orally, parenterally, pulmonarily, nasally, sublingually, lingually, buccally, rectally, transdermally, conjunctivally, otically or as an implant. Administration is preferably carried out orally.

For these administration routes, the active compound can be administered in suitable administration forms.

Those suitable for oral administration are known administration forms which release the active compound rapidly and/or in modified form, such as, for example, tablets (non-coated and coated tablets, e.g. enteric coatings), capsules, sugar-coated tablets, granules, pellets, powders, emulsions, suspensions and solutions.

Parenteral administration can be carried out with avoidance of an absorption step (intravenous, intraarterial, intracardiac, intraspinal or intralumbar) or with inclusion of an absorption (intramuscular, subcutaneous, intracutaneous, percutaneous or intraperitoneal). Suitable administration forms for parenteral administration are, inter alia, injection and infusion preparations in the form of solutions, suspensions, emulsions, lyophilizates and sterile powders.

Forms suitable for other administration routes are, for example, inhalatory pharmaceutical forms (inter alia powder inhalers, nebulizers), nasal drops/solutions, sprays; tablets or capsules to be administered lingually, sublingually or buccally, suppositories, ear and eye preparations, vaginal capsules, aqueous suspensions (lotions, shake mixtures), lipophilic suspensions, ointments, creams, milk, pastes, dusting powder or implants.

The active compounds can be converted into the administration forms mentioned in a manner per se known. This is carried out using inert non-toxic, pharmaceutically suitable excipients. These include, inter alia, vehicles (e.g. microcrystalline cellulose), solvents (e.g. liquid polyethylene glycols), emulsifiers (e.g. sodium dodecyl sulphate), dispersing agents (e.g. polyvinylpyrrolidone), synthetic and natural biopolymers (e.g. albumin), stabilizers (e.g. antioxidants such as ascorbic acid), colourants (e.g. inorganic pigments such as iron oxides) or taste and/or odour corrigents.

In general, it has proved advantageous in the case of parenteral administration to administer amounts of approximately 0.001 to 30 mg/kg, preferably approximately 0.01 to 10 mg/kg, of body weight to achieve efficacious results. In the case of oral administration, the amount is approximately 0.01 to 100 mg/kg, preferably approximately 0.1 to 30 mg/kg, of body weight.

In spite of this, if appropriate it may be necessary to depart from the amounts mentioned, namely depending on the body weight, route of administration, individual behaviour towards the active compound, manner of preparation and time or interval at which administration takes place.

### Determination of the Avonistic action on Human Recombinant 5-HT_{1A} Receptors

Signal transduction studies were carried out on human recombinant 5-HT_{1A} receptors using the guanosine 5′-O-(3-[S-35]thio)-triphosphate (GTP gamma[S-35]) binding technique (modified according to Elliott and Reynolds Europ J. Pharmacol 1999, 386, 313-315 and Sim et al. J. Neurosci 1996, 16, 8057-8066).

Repinotan hydrochloride and Example 11 in this test achieved EC₅₀ values of 0.51 nM and 0.19 nM respectively, i.e. both substances are 5-HT_{1A} agonists, Example 11 being approximately twice as potent as repinotan hydrochloride.

### MPTP Monkey Model

The in vivo action of repinotan hydrochloride and of Example 11 was tested in a monkey model of Parkinson's disease, the ‘chronic MPTP model’ (Bézard et al. Brain Res. 1997, 766, 107-112). MPTP (=1-methyl-4-phenyl-1,2,3,6-tetrahydro-pyridine) is a neurotoxin which in humans and animals causes the degeneration of dopaminergic neurons in the substantia nigra typical of Parkinson's disease. Moreover, MPTP in humans and in monkeys produces the motor symptoms typical of Parkinson's disease. These symptoms are assessed on a Parkinson scale for monkeys.

For the experiments, rhesus monkeys (Macaca fascicularis) were treated daily with MPTP (0.2 mg/kg i.v.) until they had achieved a score of 8 on the Parkinson scale. The first Parkinson symptoms occur after 5-10 days' MPTP treatment. On account of the long-term action of the neurotoxin, the clinical symptoms of the animals develop further as far as complete parkinsonism (score>15). Five groups of animals were investigated: the first received only MPTP, the second received MPTP plus repinotan hydrochloride (2 mg/kg p.o. bid), the third received Example 11 (1 mg/kg p.o. bid). The treatment with repinotan hydrochloride and Example 11 in each case began from the day on which the animals showed clinical symptoms for the first time. Both repinotan hydrochloride and Example 11 had neuroprotective activity after oral administration, i.e. both substances slowed the development of the parkinsonism symptoms in this monkey model. The observation was completely surprising, however, that Example 11 moreover also decreased the degree of severity of the symptoms, i.e. had a symptomatic action (22% reduction compared with control). Such a symptomatic action was not observed, however, with repinotan hydrochloride (cf. Table 1).

TABLE 1
Action of repinotan hydrochloride and compound 1
in the MPTP-monkey model

| | | MPTP + Repinotan | MPTP + Example 11 |
|---|---|---|---|
| Group | MPTP | HCl 2.0 mg/kg | 1.0 mg/kg |
| Slowing of the | 0 | + | + |
| development of | | | |
| Parkinsonism¹⁾ | | | |
| Symptomatology | 100% | 100% | 78% |
| on the last 3 days | | | |

¹⁾0 = No slowing of the development of parkinsonism
+ = Statistically significant delay in the attainment of Score 8

### PREPARATION EXAMPLES

### Example 1

### 2-Hydroxy-3-methoxy-benzonitrile

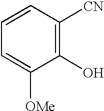

A suspension of 6375 g (41.94 mol) of o-vanillin, 3823 g (55 mol) of hydroxylamine hydrochloride and 6375 g (93.75 mol) of sodium formate in 14 l of formic acid was heated to about 90 to 95° C. with stirring. Increased evolution of gas and exothermic reaction commenced in this temperature range (heating was turned off). the exothermic reaction lasted for about 10 to 15 minutes (temperature rise to about 115° C.). The mixture was then stirred under reflux for a further 45 minutes. After completion of the reaction, the mixture was cooled to about +6° C. and stirred into a mixture of 6 kg of ice and 25 l of water. After 1 h, the solid was filtered off with suction and washed with about 12 l of water. It was then dried for 24 h at room temperature in a fresh air drying oven and for 120 h over P₂O₅ in a vacuum drying oven (room temperature).

Yield: 4816 g (77%) of crystals, m.p. 54° C., R_{f}: 0.34 (toluene-ethyl acetate 3:1)

### Example 2

2-Hydroxy-3-methoxy-acetophenone

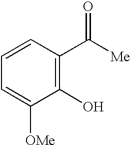

750 g (30.8 mol) of magnesium turnings and 3 g of iodine were introduced into a dry reaction vessel flushed with nitrogen. 10 l of methoxybenzene were added thereto and the mixture was warmed to 40° C. with slow stirring. The stirring was interrupted and 25 ml of methyl iodide and a starter mixture¹⁾ were added directly to the magnesium turnings. After the reaction had started, the stirrer was again switched on, and a solution of 1916 ml (30.8 mol) of methyl iodide in 2.5 l of methoxybenzene was added with moderate cooling such that it was possible to maintain a temperature of 40 to 43° C. (1.5 h). The mixture was then stirred at 40° C. for a further 5 h and at room temperature for 15 h. It was cooled to +5° C., a solution of 1840 g (12.3 mol) of 2-hydroxy-3-methoxy-benzonitrile in 6.5 l of methoxybenzene was allowed to run in in the course of 1.5 h and the mixture was stirred at 40° C. for 1.5 h. After completion of the reaction (TLC checking/toluene-ethyl acetate 3:1), the reaction mixture was cooled to +10° C. and stirred into a mixture of 24 kg of ice and 8 l of water. It was then acidified by addition of 12 l of 6N hydrochloric acid, a temperature of 0 to 5° C. not being exceeded. The organic phase was separated off and washed with 2.5 l of 6N hydrochloric acid. The combined aqueous phases were extracted 3× using 4 l of toluene each time. The aqueous phase was then stirred at an internal temperature of 98° C. for 1.5 h. The heating was turned off and 6 kg of sodium chloride were added and the mixture was stirred overnight at room temperature. 6 ¹⁾ the “starter mixture” used was the same reaction on a 1 mol scale After relatively long cooling to +5° C., a precipitate of ochre-coloured crystals was obtained. This was filtered off with suction, washed with 4 l of ice water (2×) and dried over P₂O₅ and NaOH pellets in a vacuum drying oven for 5 days (120 h).

Yield: 1587 g (78%), m.p. 53° C., R_{f}: 0.33 (toluene:ethyl acetate 9:1)

### Example 3

### Ethyl 8-methoxy-chromone-2-carboxylate

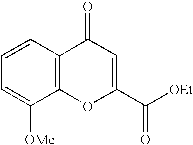

A largely dissolved mixture of 1140 g (6.86 mol) of 2-hydroxy-3-methoxy-acetophenone, 2 l (14.86 mol) of diethyl oxalate and 7 l of ethanol were added rapidly at 50° C. to a cooled solution of 1024 g (15.04 mol) of sodium ethoxide in 20 l of ethanol. The mixture was heated to reflux for 3 h. It was cooled to 50° C., 2 l of conc. hydrochloric acid were added and the mixture was heated to reflux for 30 min. It was then cooled to 50° C., the solid was filtered off with suction, the filter residue was washed with ethanol and the filtrate was concentrated in a rotary evaporator.

The yellow crystalline residue was dissolved in 15 l of dichloromethane and thoroughly stirred with 14 l of 10% strength NaHCO₃ solution for 30 minutes. The organic phase was separated off, washed with 2 l of 10% strength NaHCO₃ solution and dried over a mixture of 2 kg of Na₂SO₄ and 1 kg of tonsil. The solid was then filtered off with suction through kieselguhr, washed with dichloromethane and the filtrate was concentrated in a rotary evaporator. The residue was treated with 2.5 l of petroleum ether in the rotary evaporator and the mixture was stirred at room temperature for 15 min, cooled to +5° C. and filtered with suction. The pale yellow crystals were dried in a fresh air drying oven for 2 h and over P₂O₅ in a vacuum drying oven for 24 h.

Yield: 1304 g (76%) crystals, m.p. 129-130° C., R_{f}: 0.53 (toluene:ethyl acetate=85:15)

### Example 4

### Ethyl 8-methoxy-chroman-2-carboxylate

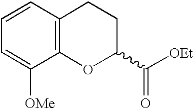

A mixture of 5.30 kg (22.45 mol) of ethyl 8-methoxy-chromoane-2-carboxylate in 100 l of ethyl acetate and 50 l of glacial acetic acid was hydrogenated at 50° C. for 24 h under a pressure of 3 bar of hydrogen in the presence of 500 g of 10% strength Pd/C. For working-up, the reaction solution was filtered with suction through kieselguhr and the filtrate was concentrated in a rotary evaporator. In order to remove residues of glacial acetic acid azeotropically, the contents of the flask were treated 2× with 6 l of toluene and concentrated. After drying the residue in a vapour-jet vacuum (8 h/8 mm), the product was obtained as a dark oil.

Yield: 5.002 kg (94%) of oil, b.p. 110-114° C./0.04 mm, R_{f}: 0.42 (toluene: ethyl acetate 3:1)

### Example 5

### (R)-2-Hydroxymethyl-8-methoxy-chroman

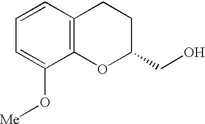

235 g of ethyl (R)-8-methoxy-chroman-2-carboxylate (obtained according to the process specified in DE-A 4430089 from ethyl (R,S)-8-methoxy-chroman-2-carboxylate) were slowly added dropwise in toluene to 540 g of 65% RedAl in toluene; a total of 1.5 l of toluene were used. After stirring at room temperature for 18 h, the mixture was heated first to 50° C. for 1 h, then to 80° C. for a further hour. After cooling to room temperature, 100 g of ice were added with external cooling, followed by 600 ml of 15% potassium sodium tartrate solution. The mixture was diluted with 500 ml of toluene and 500 ml of ethyl acetate. The organic phase was separated off, dried over magnesium sulphate and clarified by addition of tonsil. After concentrating to a volume of about 500 ml, 1 l of cyclohexane was added and the mixture was stirred at room temperature for 30 min. The precipitated solid was filtered off with suction, washed and dried. 135.5 g of target compound were thus obtained.

M.p. 77-78° C.

### Example 6

### (R)-8-Hydroxy-2-hydroxymethylchroman

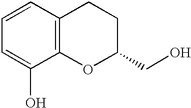

135.5 g (0.7 mol) of (−)-2-hydroxymethyl-8-methoxychroman are heated in 0.7 l of 48% strength aqueous HBr solution. After cooling and dilution with 1.2 l of ice water, the mixture is stirred for 30 minutes, and the deposited precipitate is filtered off with suction. Washing with ice water and drying over phosphorus pentoxide yield 109.5 g (87%) of the title compound, m.p. 131-132° C.

α²⁰₂₈₉=−133.8 (c=0.7 methanol)

### Example 7

### (R)-2-Hydroxymethyl-8-isopropoxy-chroman

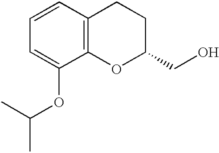

4.5 g (25 mmol) of (R)-8-hydroxy-2-hydroxymethyl-chroman, 4.6 g (27 mmol) of 2-iodopropane and 5.2 g (37.5 mmol) of powdered potassium carbonate in 50 ml of dimethylformamide are heated at 60° C. for 40 hours. After addition of a further 0.9 g of iodopropane, the mixture is heated at 80° C. for 24 hours and then at 95° C. for a further 24 hours. After cooling, it is partitioned between toluene/water and filtered through Celite®. The organic phase is dried (magnesium sulphate) and concentrated. After flash chromatography (silica gel; elution with toluene/ethyl acetate gradients 3:1-2:1), 7 g of crude product are obtained, which is purified by chromatography on silica gel (gradient toluene/ethyl acetate 1:0-8:1). Yield: 2.9 g (52%) of oil.

R_{F} (silica gel, toluene/ethyl acetate 1:1): 0.4 [α]²⁰₂₈₉=−85[c=0.5; CHCl₃]

### Example 8

### (R)-8-Isopropoxy-2-mesyloxymethyl-chroman

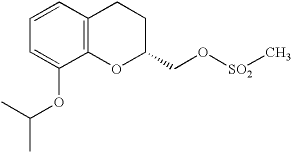

68 g (0.6 mol) of methanesulphonyl chloride are added dropwise at room temperature to 114 g (0.51 mol) of the compound from Example 7 in 95 g of pyridine. After stirring at room temperature for 18 hours, the mixture is diluted with 700 ml of water and extracted with dichloromethane. Filtration through silica gel and concentration afford 150 g of crude product, which is purified by crystallization from 1.5 l of cyclohexane/toluene mixture 3:1. The mother liquor is recrystallized from cyclohexane after concentration. 112 g of title compound are thus obtained as a colourless solid, m.p. 77-78° C.

□₂₈₉²⁰=−56.2 [c=0.9, CH₃OH]

### Example 9

### (R)-2-Benzylaminomethyl-8-isopropoxy-chroman

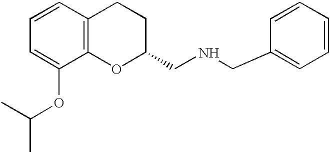

112 g (0.37 mol) of the compound from Example 8, 200 g (1.87 mol) of benzylamine and 3.0 g (0.02 mol) of sodium iodide are heated at 100° C. for 5 hours. After cooling, the solid is separated off and the organic phase is washed 2× with 2.5 l of water each time. The residual oil is taken up with 1 l of ethyl acetate. Washing the ethyl acetate phase with water and saturated sodium chloride solution and subsequent drying and concentration afford 114.5 g (quant.) of the title compound (HPLC purity: 93%) as an oil which is employed in the next stage.

□₂₈₉²⁰=−104[c=0.5, CH₃OH]

### Example 10

### (R)-2-(N-Benzyl-N-(4-(1,1-dioxido-3-oxo-2,3-dihydro-benzisothiazol-2-yl)-2-butinyl)-aminomethyl)-8-isopropoxy-chroman hydrochloride

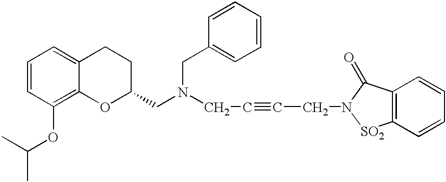

114 g (0.37 mol) of the compound from Example 9 and 13.5 g (0.45 mol) of paraformaldehyde in 1 l of dioxane are treated with 4 g of copper(II) acetate and warmed to 50° C. 81 g (0.37 mol) of propargylsaccharin are added at this temperature. After stirring at 80° C. for 2 hours, the mixture is concentrated and the residue is partitioned between toluene/water with addition of tonsil. After filtration of the mixture through Celite®, the organic phase is separated off and purified by flash chromatography on silica gel (toluene/ethyl acetate 10:1). Precipitation of the hydrochloride from ether using ethereal hydrochloric acid affords 226 g of crude product. After liberation of the free base using sodium hydrogencarbonate, this product is purified by chromatography on silica gel (elution with toluene/ethyl acetate 20:1). The product fractions are treated with ethereal hydrochloric acid. 139 g (65%) of title compound are thus obtained as a solid, m.p. 106-109° C.

□₂₈₉²⁰=−64.1[c=0.8, CH₃OH]

### Example 11

2-[4-({[(2R)-8-Isopropoxy-chroman-2-yl]methyl}amino)butyl]-1,2-benzisothiazol-3(2H)-one 1,1-dioxide has the following structure:

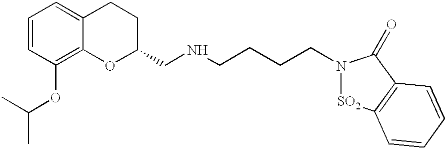

120 g (0.21 mmol) of the compound from Example 10 in in 1.4 l of methanol are treated with 400 ml of conc. hydrochloric acid and 20 g of 10% palladium on active carbon. After hydrogenating at normal pressure and 20° C. for 4 hours, the catalyst is filtered off and the filtrate is concentrated. The residue is concentrated 2× with toluene and dissolved using 400 ml of ethyl acetate. Addition of 800 ml of diethyl ether and stirring at room temperature for 18 h afford 90.5 g of solid after filtering off with suction and drying in vacuo. Recrystallization from 1 l of acetonitrile and washing the crystals with diethyl ether afford 70.8 g (69%) of title compound as colourless crystals, m.p. 153-154° C.

□₂₈₉²⁰ = 65.9 [c = 0.6, CH₃OH]

| Elemental analysis: | | C₂₄H₃₀N₂O₅S × HCl | | | | |
|---|---|---|---|---|---|---|
| calc.: | C: 58.2 | H: 6.3 | N: 5.7 | O: 16.2 | Cl: 7.2 | S: 6.5 |
| found: | C: 58.0 | H: 6.3 | N: 5.7 | O: 16.2 | Cl: 7.1 | S: 6.3 |

1. Use of 2-[4-({[(2R)-8-isopropoxy-chroman-2-yl]methyl}amino)butyl]-1,2-benzisothiazol-3(2H)-one 1,1-dioxide, its physiologically acceptable salts, hydrates and/or solvates for the production of a medicament for the prophylaxis and/or treatment of Parkinson's disease.

2. Use according to claim 1, the active compound being 2-[4-({[(2R) -8-isopropoxy-chroman-2-yl]methyl}amino)butyl]-1,2-benzisothiazol-3(2H)-one 1,1-dioxide hydrochloride.

3. 2-[4-({[(2R)-8-Isopropoxy-chroman-2-yl]methyl} amino)butyl]-1,2-benziso-thiazol-3(2H)-one 1,1-dioxide, its physiologically acceptable salts, hydrates and/or solvates for the prophylaxis and/or treatment of Parkinson's disease.

4. 2-[4-({[(2R)-8-Isopropoxy-chroman-2-yl]methyl}amino)butyl]-1,2-benziso-thiazol-3(2h)-one 1,1-dioxide hydrochloride for the prophylaxis and/or treatment of parkinson's disease:
